(19) United States
(12) Patent Application Publication
(10) Pub. No.: US 2016/0115436 A1
(43) Pub. Date: Apr. 28, 2016

(54) DIGITAL MICROFLUIDIC PLATFORM FOR CREATING, MAINTAINING AND ANALYZING 3-DIMENSIONAL CELL SPHEROIDS

(71) Applicant: THE REGENTS OF THE UNIVERSITY OF CALIFORNIA, Oakland, CA (US)

(72) Inventors: Andrew Aijian, Los Angeles, CA (US); Robin L. Garrell, Los Angeles, CA (US)

(73) Assignee: The Regents of the University of California, Oakland, CA (US)

(21) Appl. No.: 14/889,926

(22) PCT Filed: May 12, 2014
(86) PCT No.: PCT/US2014/037706

§ 371 Date: Nov. 9, 2015

Related U.S. Application Data
(60) Provisional application No. 61/821,874, filed on May 10, 2013.

(51) Int. Cl. C12M 1/42; C12M 1/00; C12M 3/06

(52) U.S. Cl. CPC C12M 35/02; C12M 23/16; C12M 23/20; C12M 29/20

(57) ABSTRACT

The invention provides a microfluidic system for forming and/or analyzing multi-cellular spheroids in a hanging drop cell culture. Embodiments of the invention include microfluidic (DμF) systems capable of creating and supporting hanging droplets of cell culture media for the purpose of initiating and maintaining the growth of three-dimensional, multi-cellular spheroids. The microfluidic systems disclosed herein are compatible with numerous analysis modalities including microscopy, mass spectrometry, and fluorescence spectroscopy.

### CROSS REFERENCE TO RELATED APPLICATIONS

This application claims the benefit under 35 U.S.C. Section 119(e) of co-pending U.S. Provisional Patent Application Ser. No. 61/821,874, titled “DIGITAL MICROFLUIDIC PLATFORM FOR CREATING, MAINTAINING AND ANALYZING 3-DIMENSIONAL CELL SPHEROIDS” filed May 10, 2013, the contents of which are incorporated herein by reference.

### STATEMENT AS TO RIGHTS TO INVENTIONS MADE UNDER FEDERALLY SPONSORED RESEARCH AND DEVELOPMENT

This invention was made with Government support under Grant Nos. DGE0114443 and DGE0654431, awarded by the National Science Foundation. The Government has certain rights in the invention.

### TECHNICAL FIELD

The invention relates to microfluidic systems and methods for creating, maintaining and analyzing three-dimensional, multi-cellular spheroids.

### BACKGROUND OF THE INVENTION

Cell spheroids are multi-cellular, compact aggregates of cells grown in-vitro that possess a three-dimensional (3D), spherical morphology. Unlike cells grown in two-dimensional (2D) monolayers, cells grown in three dimensions possess a high degree of intercellular interactions and exhibit relatively complex nutrient and metabolic transport profiles, leading to cellular heterogeneity within the 3D aggregate as well as gene and protein expression patterns that more closely mimic in-vivo tissues (see, e.g. T. W. Ridky et al., Nat Med, 2010, 16, 1450-1455; G. R. Souza et al., Nature Nanotechnology, 2010, 5, 291-296; A. Birgersdotter et al., Leuk Lymphoma, 2007, 48, 2042-2053; P. De Witt Hamer et al., Oncogene, 2008, 27, 2091-2096; A. Ernst et al., Clin Cancer Res, 2009, 15, 6541-6550; N. C. Cheng et al., Stem Cells Transl Med, 2013, 2, 584-594). These differential expression profiles lead to significant differences in cellular properties (e.g. drug sensitivity, differentiation capacity, malignancy, function, and viability) for cells cultured in monolayers compared to three dimensions. For example, hepatocellular carcinoma cells grown as spheroids exhibit more physiologically relevant levels of cytochrome P450 activity and albumin secretion compared to monolayer cells (see, e.g. T. T. Chang et al., Tissue Eng Part A, 2009, 15, 559-567). In another example, mammary epithelial cells exhibit basement membrane-induced apoptosis resistance when grown in three dimensions but are susceptible to apoptosis in monolayer culture (see, e.g. N. Boudreau et al., Proc Natl Acad Sci USA, 1996, 93, 3509-3513). Thus, due to their three-dimensional morphology and high degree of intercellular interactions, cell spheroids are able to provide a more physiologically relevant model of tissues than monolayer cells. Furthermore, this enhanced physiological relevance allows cell spheroids to provide a more accurate cellular model for cell-based assays and screens.

Despite the well-known advantages of three-dimensional cell cultures, the use of 3D cell models in cell-based assays and screens has been limited. It is estimated that less than 30% of cancer and molecular biologists utilize 3D cell cultures and that less than 20% of drug leads generated by the pharmaceutical industry are done so using cell-based phenotypic assays (see, e.g. D. W. Hutmacher, Nat Mater, 2010, 9, 90-93; J. A. Lee et al., J Biomol Screen, 2013, 18, 1143-1155). One major reason for the relatively low adoption of 3D cell models is the limited number of user-friendly, flexible, and automated methods for performing spheroid culture and analysis (see, e.g. W. Y. Ho et al., Plos One, 2012, 7, e44640; L. Kunz-Schughart et al., J Biomol Screen, 2004, 9, 273-285). Current multi-cellular spheroid creation technologies typically rely on using: (a) non-adhesive surfaces or micromolds to make numerous spheroids simultaneously (see, e.g. Scivax USA Inc., Microtissues Inc., Transparent Inc.); (b) specialized well-plates that are compatible with robotic liquid handling systems to generate and assay large numbers of spheroids (see, e.g. InSphero AG, 3D Biomatrix); or (c) hydrogel or ECM molecules/materials to encapsulate the cells in a three-dimensional environment (see, e.g. Cellendes, Neuromics). Another approach utilizes magnetic assisted levitation to suspend cells and induce spheroid formation (see, e.g. n3D Biosciences Inc., Hamilton Company). Rotary culture systems available from various manufacturers are also used in the formation of three dimensional cell spheroids.

While various technologies and methods are available for the culturing of three-dimensional micro-tissues, each approach has limitations making it unsuitable for routine assaying and screening (see, e.g. R.-Z. Lin et al., Biotechnol J, 2008, 3, 1172-1184). These methods are limited, for example, by tedious manual pipetting protocols, the necessity of robotic liquid handling equipment or the inability to assay individual spheroids. For instance, non-automated methods often require a significant amount of manual sample handling, which can be tedious, time-consuming, and prone to variability and error. Though inexpensive and relatively simple to perform, manual spheroid formation techniques and micromold methods require manually harvesting and transferring the spheroids individually into separate containers such as microplates for analysis. Rotary vessels and spinner flasks can be used to generate a large number of spheroids, but provide limited control over spheroid size and do not allow for in-situ assaying of individual spheroids.

Alternatively, specially engineered well plates, such as those capable of supporting hanging drop culture or those with non-adhesive surfaces designed to induce cell aggregation, are compatible with robotic liquid handling equipment, which allows for automation, in-situ assaying, and high-throughput processing. However, such robotic liquid handling systems are expensive to acquire and maintain, complicated to operate, troubleshoot, and repair, and require relatively large sample and reagent volumes. These systems also lack the ability to reconfigure assay protocols in real-time. Therefore, robotic liquid handlers are effective for performing simple high-throughput liquid handling operations, but are not economically or functionally practical for researchers who seek assay flexibility and do not require high-throughput capabilities.

Thus, it is clear that there is a need for a spheroid culture and analysis technology that can provide the advantages of automation in a platform that is more accessible than the currently existing automation methods. In particular, there is a need for a three-dimensional cell-culture technology that can provide complete automation of culture and analytical protocols combined with assay flexibility, without the need for expensive and complex robotic liquid handling equipment.

### SUMMARY OF THE INVENTION

The present invention addresses the above-mentioned needs and provides further advantages over conventional cell culture systems by using a droplet microfluidic system that can form and/or maintain and/or analyze multi-cellular spheroids in a hanging drop culture. In illustrative embodiments of the invention, a digital microfluidic (DμF) system is used to create and support hanging droplets of cell culture media for the purpose of initiating and maintaining the growth of three-dimensional, multi-cellular spheroids of mammalian cells. One or many spheroids may be created, maintained, and analyzed on a single device. These digital microfluidic systems enable the real-time analysis of the spheroids or molecules secreted by the spheroids, and are designed to be compatible with numerous analysis modalities including microscopy, mass spectrometry, and fluorescence spectroscopy. Embodiments of the digital microfluidic systems disclosed herein include a relatively low-cost platform with automated, precise, and flexible liquid handling capabilities, one which provides a more accessible alternative to existing culture automation techniques for multi-cellular spheroids of mammalian cells.

As noted above, the invention provides droplet microfluidic systems useful for forming and/or analyzing multi-cellular spheroids in a hanging drop culture as well as method for making and using such systems. An illustrative embodiment of the invention is a microfluidic cell culture system comprising a first plate, a second plate parallel to and facing/opposite the first plate, an array of electrodes disposed on the first plate or the second plate; and a well disposed on the first or second plate. In this system, the elements are arranged in a three dimensional constellation of elements designed so that that when an electric potential is applied to the array of electrodes, a droplet of liquid cell culture media within the system can be moved along the array of electrodes and to the well, and further be drawn into the well by capillary forces.

A variety of illustrative embodiments of the invention are disclosed herein (see, e.g. those disclosed in FIGS. 3, 4, 11 and 17). In some embodiments of the invention, the first plate or the second plate in the microfluidic system is coated with a hydrophobic material disposed on the plate in region(s) selected to facilitate movement of the droplet of liquid cell culture media through the system. In certain embodiments of the invention, the well is coated with a hydrophilic material in region(s) selected to facilitate movement of the droplet of liquid cell culture media into the well. Optionally, the well comprises an open lower end, so that a bottom portion of the droplet of liquid cell culture media is suspended and does not contact a surface. In some embodiments of the invention, the diameter well is greater than or equal to 2.4 mm and/or the Bond number of the system is greater than or equal to 0.3. Optionally, the well comprises a material such as an oil selected for its ability to coat the droplet of liquid cell culture media drawn into the well, thereby providing this droplet with a protective coating against evaporation.

In typical embodiments of the invention, the array of electrodes is arranged within the microfluidic system in a three dimensional architecture designed so that a sequential application of an electric potential to the array of electrodes controls the movement of the droplet of liquid cell culture media within the system. In illustrative embodiments of the invention, the array of electrodes comprises an actuating electrode and a ground electrode; and the actuating electrode is disposed on the first plate and the ground electrode is disposed on the second plate. In certain embodiments of the invention, system further comprises one or more of ports adapted to introduce droplets of liquid cell culture media to the system, a humidity reservoir disposed under the well, a ventilation conduit through the first plate or the second plate, and/or a spacer that separates the first or the second plate at a defined distance (see, e.g. FIG. 17). Typically, the microfluidic system comprises one or more automation elements such as a processor adapted to facilitate the sequential application of electric potentials to the array of electrodes.

Other illustrative embodiments of the invention comprise methods of forming a spheroid mammalian cell culture within a droplet of cell culture media. These methods typically comprise first providing a microfluidic system as disclosed herein, one which includes for example, a first plate; a second plate parallel to and opposite/facing the first plate; an array of electrodes disposed on the first or second plate; and a well on the first or second plate (e.g. one comprising a hydrophilic surface). Optionally in these methods, the well comprises an open lower end, so that a bottom portion of the droplet of cell culture media is suspended and does not contact a surface; and/or the well comprises a convex surface that contacts and stabilizes droplets of cell culture media. In some embodiments of the invention, the diameter of the well is greater than or equal to 2.4 mm.

Various illustrative aspects of the present invention are shown, for example, in FIGS. 3-8, 11 and 17. Typically these microfluidic systems comprise a first plate and a second plate parallel to and opposite/facing the first plate, an array of electrodes disposed on the second plate, and a well on the first or second plate. In such systems, a droplet of liquid can be moved along the array of electrodes to the well such that the droplet of liquid is drawn into the well by capillary forces. In common embodiments, the array of electrodes is coated with a dielectric insulating material. In one or more embodiments of the invention, the well does not have a bottom, thereby allowing the droplet of liquid to be suspended such that the bottom of the droplet does not touch a solid surface. In other embodiments, the well comprises a round bottom.

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 illustrates several current spheroid formation methodologies. FIG. 1a shows the manual pipetting of drops of cell solution onto a surface and inversion of the surface to create hanging drops. This method is labor intensive and time consuming. FIG. 1b shows the addition of cell solution to a surface containing an array of non-adhesive micro-wells which allows formation of large numbers of spheroids. This method requires manually transferring of the spheroids into a well-plate in order to assay them individually, which is a tedious process. FIG. 1c shows a modified well-plate (top) which allows hanging drops to be formed automatically using a robotic liquid handling system (bottom). This method requires expensive and complex equipment as well as relatively large sample and reagent volumes.

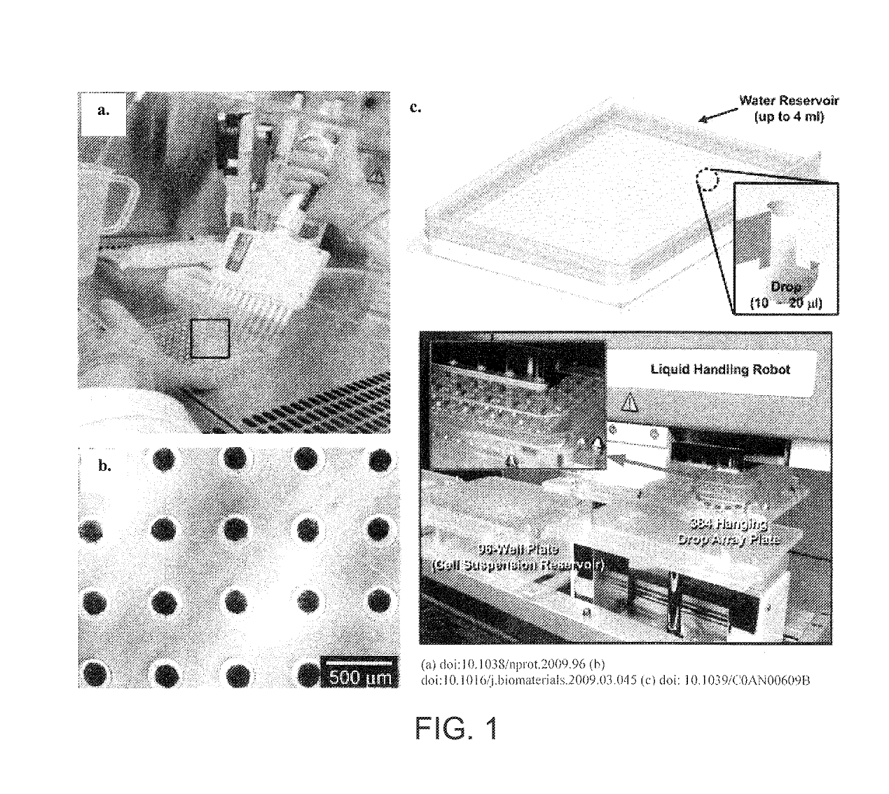

FIG. 2 illustrates example benchtop systems manufactured by Advanced Liquid Logic, Inc. (Morrisville, N.C.) for biomolecular analyses using digital microfluidics. These systems contain integrated thermal control, optical detection, magnetic actuators, computer processing, a touch-screen interface, and utilize disposable cartridges.

FIG. 3 is a schematic showing how a digital microfluidic device enables spheroid culture and analysis by supporting (a) a hanging drop culture or (b) a microwell culture of spheroid-forming cells.

FIG. 4 illustrates a digital microfluidic device that supports hanging drops through the incorporation of through-holes or ‘wells’ into the electrode footprints, in accordance with one embodiment of the present invention.

FIG. 5 illustrates (a) an assembled digital microfluidic device, in accordance with one embodiment of the present invention, consisting of a top plate, spacer, and bottom plate. The through-holes in the top plate allow solutions to be added to the device. Droplets can be moved along the array created by the actuation electrodes to the locations of the wells in the bottom plate where they can form hanging drops in the space below the bottom plate (b).

FIG. 6 illustrates a setup for operating a digital microfluidic device, in accordance with one embodiment of the present invention. The device is connected to an aluminum plate that sits on a top plate. A window machined within the plate allows hanging droplets to form beneath the bottom plate of the device. The device is connected to a cable that allows a user to control and program droplet actuation using a computer. The hot plate allows the device to be kept at a temperature suitable for cell culture.

FIG. 7 is a sequence of images showing the dispensing of a droplet of Liebovitz L-15 medium from the reservoir and the insertion of the droplet into a well on a digital microfluidic device to form a hanging drop. The drop is dyed blue to aid in visualization.

FIG. 8 is a series of time-lapse images showing cell aggregation within a hanging drop on the device over the course of the initial 24 hours after drop formation. At 24 hours after drop formation, the cells aggregate into a 200-μm diameter spheroid. Engulfing the hanging drop in non-volatile oil prevents any significant droplet evaporation, allowing for long-term hanging drop culturing.

FIG. 9 illustrates aggregates of mouse mesenchymal stem cells formed within a hanging drop over (a) 24 and (b) 48 hours. Cells were maintained in a drop of Liebovitz L-15 medium containing 10% FBS, 1% penicillin/streptomycin, and 0.02% Pluronics® F-68. The drop was engulfed in sterilized silicone oil to prevent evaporation. The cells are stained with a dye to indicate living (green) and dead (red) cells.

FIG. 10 shows an embodiment of illustrative computer system elements that can be adapted for use with embodiments of the invention.

FIG. 11 illustrates a digital microfluidic device schematic and dimensions, in accordance with one embodiment of the present invention. Indium Tin Oxide (ITO) is used for all electrodes. Through-holes in the top plate allow for the addition of solutions to on-chip reservoirs, while through-holes in the bottom plate allow for the formation of hanging drops. Drops are drawn into the well spontaneously upon contact with the hydrophilic walls of the well. In another embodiment of the device, the plates are switched so that the actuating electrode plate is on the bottom and contains the through-holes into which the drops are delivered.

FIG. 12 illustrates a hanging drop formation on a digital microfluidic device. (a) A series of images showing a top-down view of the insertion of drops of cell media (dyed blue for enhanced visualization, ˜1.2 uL) into a well on a digital microfluidic device. (b) A series of images showing a side-view of a well after the addition of multiple drops to the well. The drops are spontaneously inserted into the well and, after a sufficient volume has been added, form a hanging drop with the curved interface necessary to induce cell aggregation.

FIG. 13 illustrates the degree of medium exchange for a predicted model and experimental results of one embodiment of the invention. The degree of medium exchange after one and two exchange cycles was monitored by measuring the change in absorbance of the dyed hanging drop solution and calculating the concentration from a standard curve. The dilution of a hanging drop after each cycle can be seen in the images above the plot. The agreement between the theoretical and the experimental results indicates that thorough mixing of the hanging drop is achieved during each exchange cycle. Error bars indicate the standard deviation of measurements from three different experiments.

FIG. 14 illustrates cell spheroids after digital microfluidics hanging drop culture, showing representative images of spheroids grown on a digital microfluidic device after 24, 48, and 72 hours of in-situ incubation. Each image corresponds to a different spheroid. Spheroids exhibit viability greater than 90% during this time-frame as determined by staining with calcein-AM/ethidium homodimer-1 to visualize living (green) and dead (red) cells. Spheroids had a diameter of 280±35 μm after 24 hours (N=8) and 311±40 μm after 48 hours (N=5), and 337±31 (N=5) after 72 hours in culture.

FIG. 15 illustrates in-situ induction of spheroid adipogenesis. Representative images of spheroids of mouse mesenchymal stem cells grown in either normal (a) or adipogenic (b) conditions. To induce adipogenesis, the medium of a spheroid grown in normal conditions (containing standard growth medium) was exchanged for adipogenic medium (growth medium containing insulin and dexamethasone) after 24 hours. The spheroid was maintained in the adipogenic medium for an additional 48 hours after which it was harvested from the device, stained with the lipophilic fluorescent dye Nile Red to identify intracellular lipid (fat) droplets, and imaged on a confocal laser scanning microscope. The spheroid grown in the adipogenic conditions exhibits ˜57% more lipid content (green fluorescence) than a spheroid grown under normal conditions after equal incubation periods in their respective media.

FIG. 16 illustrates in-situ observation of spheroid aggregation. These images show a time series of cells aggregating within a well at various points after cell seeding over the course of 24 hours. At 24 hours, the cells form a single, compact spheroid within the well. The transparent nature of the device allows wells and spheroids to be imaged in real-time in-situ. Microscopic analysis can be used to track various spheroid properties, such as formation, growth, or invasion, over extended periods of time as long as the device is maintained at optimal culture conditions during the imaging.

FIG. 17 illustrates (Top) a cross-section schematic of the digital microfluidic setup, in accordance with one embodiment of the present invention. The schematic shows how the devices were assembled to enable hanging drop formation and culture. The schematic is not drawn to scale. Dimensions are provided for reference. (Bottom) A top-down schematic of an electrode configuration used for manipulating droplets. The schematic illustrates how hanging droplets are situated in wells to enable spheroid formation. The devices used in this work allow for the formation of up to 8 spheroids.

FIG. 18 illustrates a spheroid formed out of human colon carcinoma cells (HT-29 cell line). The spheroid was stained with calcein-AM and ethidium homodimer-1 to indicate living (green) and dead (red) cells, respectively. The spheroid was initiated and maintained on a digital microfluidic device for 72 hours. Medium exchange was performed after 24 and 48 hours. The spheroid grew to ˜550 μm in diameter and exhibited a center region of necrotic cells, surrounded by a rim of proliferating cells, consistent with spheroids formed by other techniques. A spheroid with a necrotic core closely resembles the hypoxic regions of tumors.

FIG. 19 is a series of images showing cross-sections of the spheroid in FIG. 18 at various heights (˜3 μm intervals) from the bottom of the spheroid (z=0 μm). The images clearly show the inner necrotic core surrounded by viable cells at various locations within the spheroid.

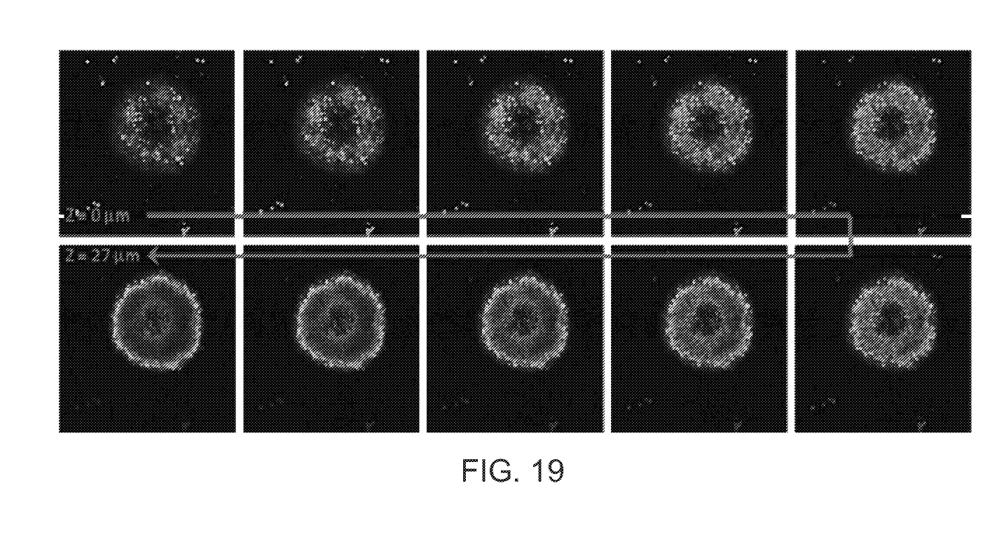

FIG. 20 is a series of images showing the movement of a collagen solution on a digital microfluidic device. The liquid regions in the images above are outlined with white dashed lines to enhance visualization of the drops. Collagen solution (here, 1 mg/mL) can be dispensed from on-chip reservoirs (1), moved to the location of a well (2), and inserted into the well (3). The addition of multiple drops of collagen solution to the well allows for the formation of a hanging drop. Incubating the device at 37° C. causes the collagen solution to gel within the wells, forming a gel-drop within the wells (4). Cell suspensions of multi-cellular spheroids can be grown inside of a gel on a digital microfluidic device. The capability to form hanging drops of hydrogels on a digital microfluidic device is useful for performing various assays, such as migration assays, or studying the role of the cellular microenvironment on cell growth and behavior.

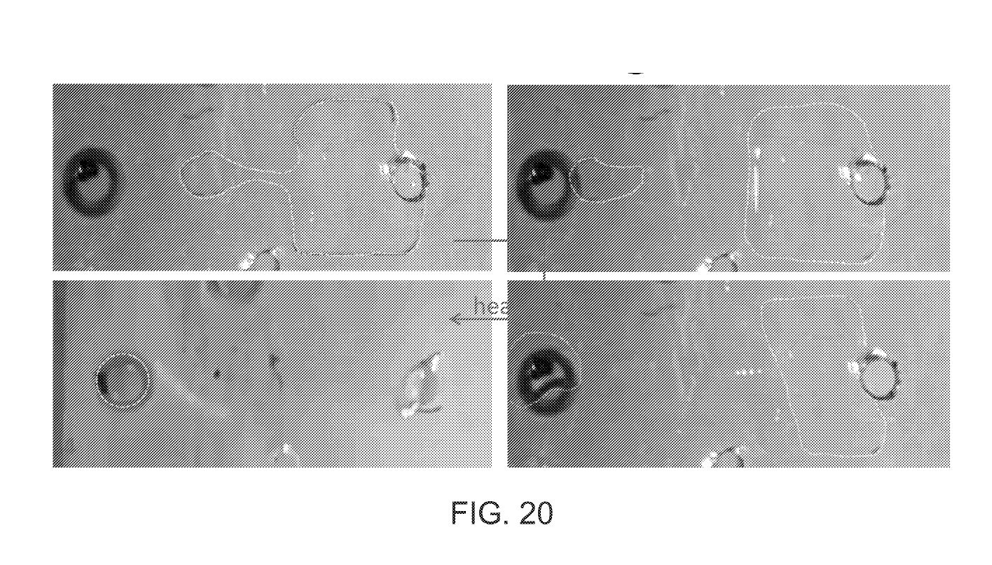

### DETAILED DESCRIPTION OF THE INVENTION

In the description of the preferred embodiment, reference is made to the accompanying drawings which form a part hereof, and in which is shown by way of illustration a specific embodiment in which the invention may be practiced. It is to be understood that other embodiments may be utilized and structural changes may be made without departing from the scope of the present invention. The present disclosure references a number of different publications as indicated throughout the specification by one or more reference numbers within brackets, e.g., (x). Each of these publications is incorporated by reference herein. Unless otherwise defined, all terms of art, notations and other scientific terms or terminology used herein are intended to have the meanings commonly understood by those of skill in the art to which this invention pertains. Many of the techniques and procedures described or referenced herein are well understood and commonly employed using conventional methodology by those skilled in the art.

Studies have shown that digital microfluidics can be used to automate two-dimensional (monolayer) cell cultures (see, e.g. I. Barbulovic-Nad et al., Lab Chip, 2010, 10, 1536-1542; Vergauwe et al., Journal of Micromechanics and Microengineering, 2011, 21.5: 054026; S. C. Shih et al., Biosens Bioelectron, 2013, 42, 314-320; I. A. Eydelnant et al., Lab Chip, 2012, 12, 750-757) and form thin hydrogel posts for scaffold-based 3D cell cultures (see, e.g. S. M. George et al., presented in part at the 15th International Conference on Miniaturized Systems for Chemistry and Life Sciences, Seattle, Wash., USA, Oct. 2-6, 2011, 2011). Studies have also described various methods for the fabrication of digital microfluidic devices (see, e.g. A. P. Aijian et al., Lab Chip, 2012, 12, 2552-2559). U.S. Patent Pub. No. 2010/0311599 describes a method using digital microfluidics to culture and assay adherent cells and cell suspensions, but does not describe a device or process that enables three-dimensional cell-culturing on a digital microfluidic device. The instant invention overcomes a number of limitations in conventional digital microfluidic systems used to culture cells.

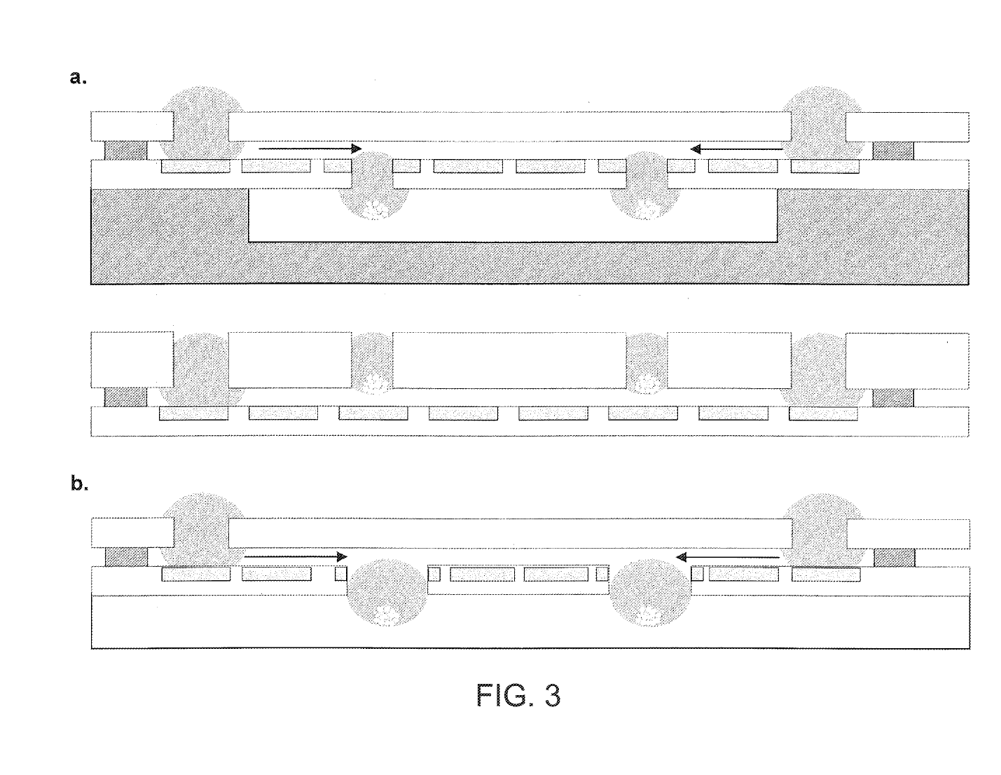

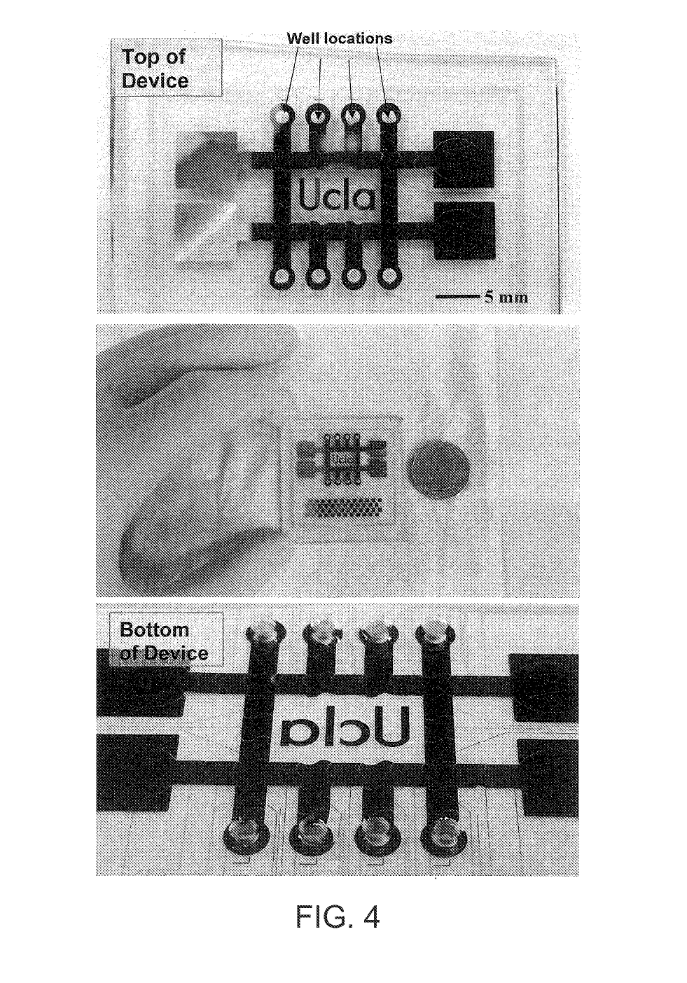

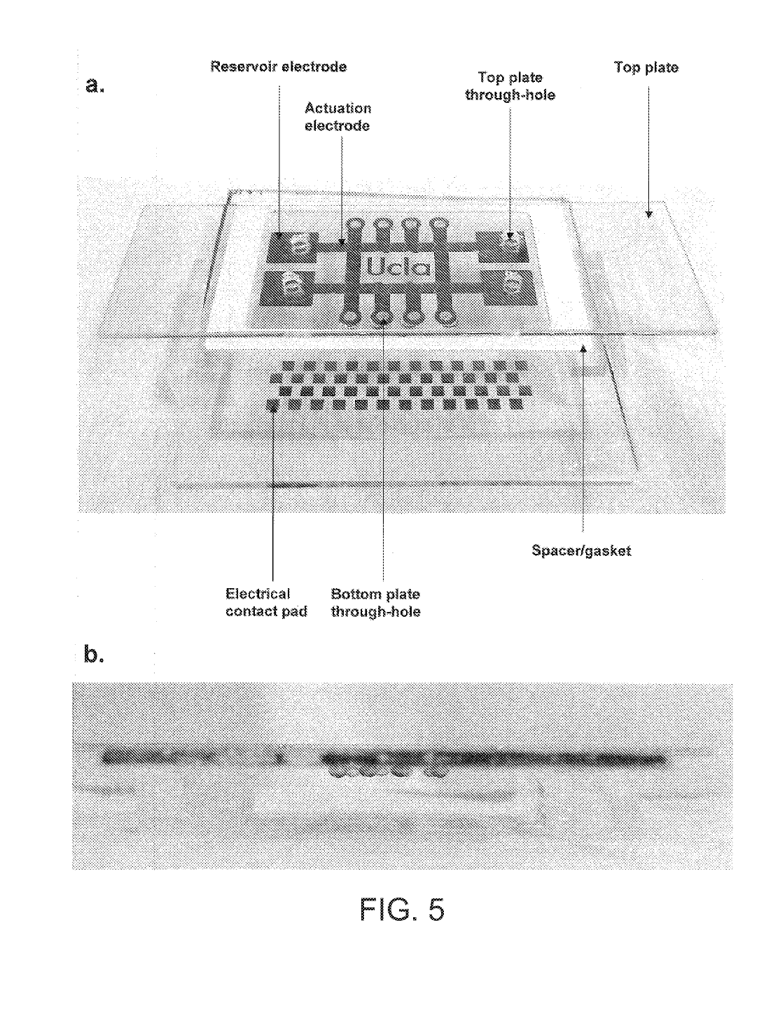

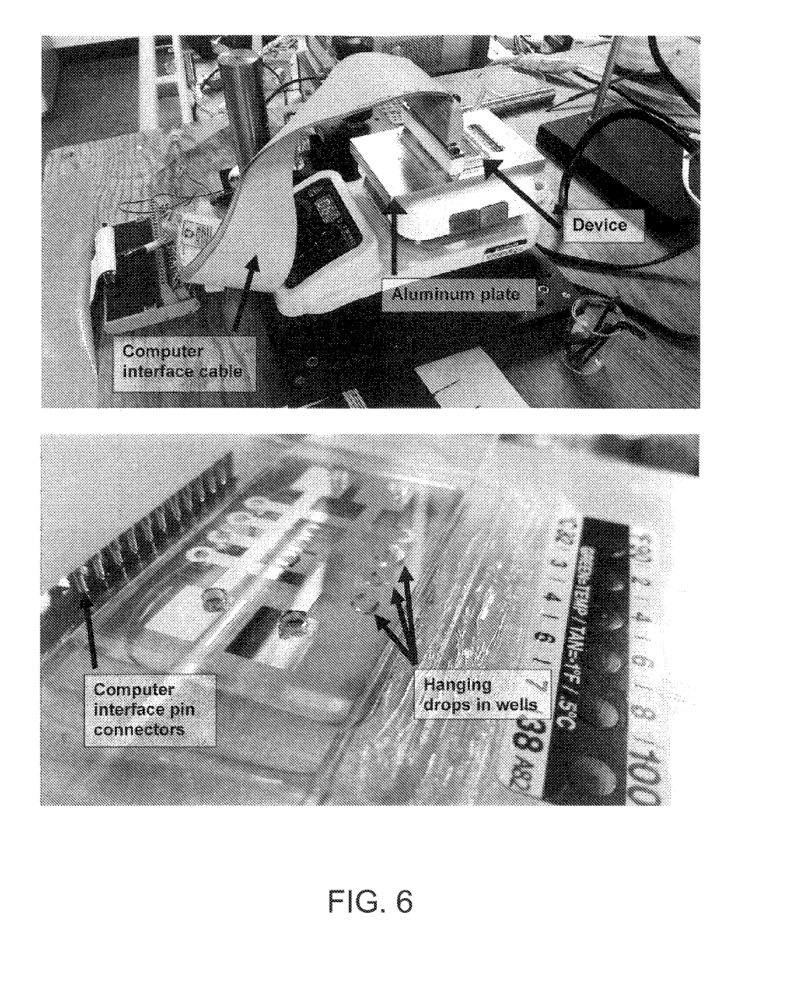

The invention disclosed herein provides droplet microfluidic systems useful for forming and/or analyzing multi-cellular spheroids in a hanging drop culture as well as method for making and using such systems. A number of illustrative embodiments of the invention are disclosed herein (see, e.g. FIGS. 3-8, 11 and 17). One illustrative embodiment of the invention is a microfluidic cell culture system comprising a first plate, a second plate parallel to and facing/opposite the first plate, an array of electrodes disposed on the first plate or the second plate; and a well disposed either the first or second plate. In this system, the elements are arranged in a three dimensional constellation of elements designed so that that when an electric potential is applied to the array of electrodes, a droplet of liquid cell culture media within the system can be moved along the array of electrodes and to the well, and further be drawn into the well by capillary forces. In certain embodiments of the invention, the droplet of liquid cell culture media comprises a spheroid of growing mammalian cells that is at least 10 μm, 100 μm or 1000 μm in diameter.

In some embodiments of the invention, the first plate or the second plate in the microfluidic system is coated with a hydrophobic material disposed on the plate in region(s) selected to facilitate movement of the droplet of liquid cell culture media through the system. In certain embodiments of the invention, the well is further coated with a hydrophilic material in region(s) selected to facilitate movement of the droplet of liquid cell culture media into the well. However, the well does not necessarily need to be coated with a hydrophilic material. In other embodiments, the plate can consist of a naturally hydrophilic substrate that is coated with a hydrophobic material except in the location of the wells. Thus, the hydrophilicity may come from the natural properties of the material and not from some additional coating. Optionally, the well comprises an open lower end, so that a bottom portion of the droplet of liquid cell culture media is suspended and does not contact a surface. In some embodiments of the invention, the diameter well is greater than or equal to 2.4 mm and/or the Bond number of the system is greater than or equal to 0.3. Optionally, the well comprises a material such as an oil selected for its ability to coat the droplet of liquid cell culture media drawn into the well, thereby providing this droplet with a protective coating against evaporation.

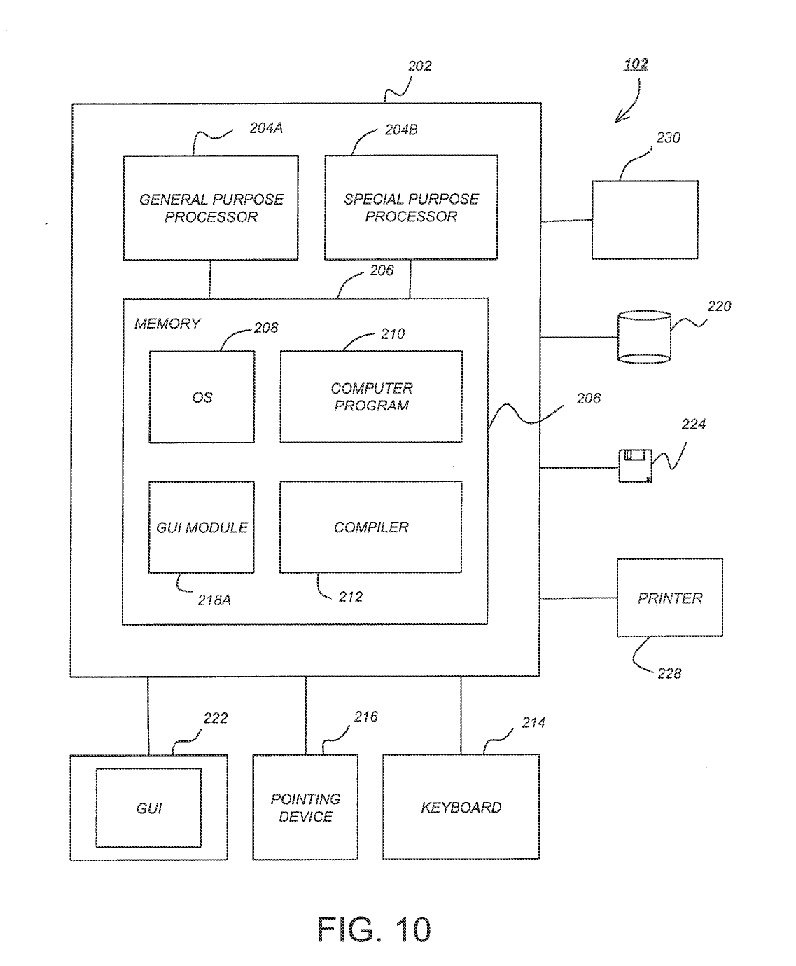

In typical embodiments of the invention, the array of electrodes is arranged within the microfluidic system in a three dimensional architecture designed so that a sequential application of an electric potential to the array of electrodes controls the movement of the droplet of liquid cell culture media within the system. In some embodiments of the invention, the array of electrodes comprises an actuating electrode and a ground electrode; and the actuating electrode is disposed on the first plate and the ground electrode is disposed on the second plate. Typically, the microfluidic system comprises one or more elements that facilitate the automation of the systems such as a processor adapted to sequentially apply electric potentials to the array of electrodes (see e.g. FIG. 10). Such elements are useful in automated embodiments of the invention, for example, automated microfluidic systems that are designed to minimize the contact between a cell culture media and the external environment (by minimizing user contact/interaction), thereby addressing problems that can result from the microbial contamination of cell culture media.

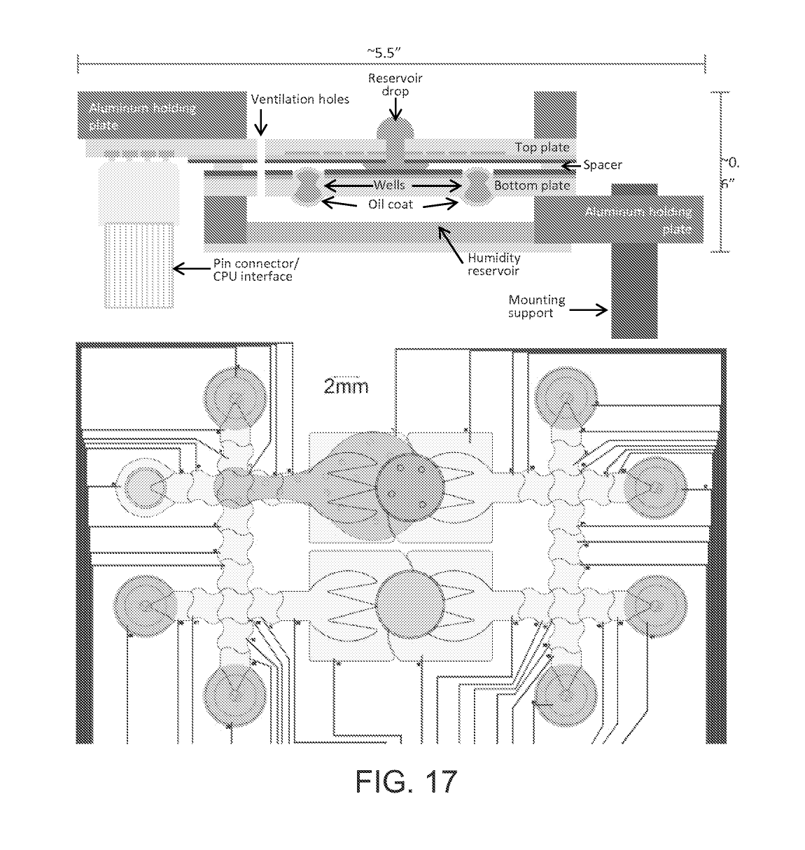

In certain embodiments of the invention, system further comprises one or more of a ports/reservoir drops adapted to introduce droplets of liquid cell culture media to the system, and/or a humidity reservoir disposed under the well, and/or a ventilation conduit through the first plate or the second plate, and/or a spacer that separates the first or the second plate at a defined distance (see, e.g. FIG. 17). In some embodiments of the invention, an array of electrodes is disposed in the system and in operable contact with a droplet introducing port so that droplet(s) of liquid introduced into a port can move in a first direction, and also a second direction (see, e.g. FIG. 17). In certain embodiments of the invention, the microfluidic system comprises a plurality of ports adapted to introduce droplets of liquid cell culture media to the system (see, e.g. FIGS. 3 and 17) and operably connected to the array of electrodes so that a first droplet of liquid introduced into first port can move along the array of electrodes in a first direction (to form a first fluid conduit) and a second droplet of liquid introduced into second port can move along the array of electrodes in a second direction (to form a second fluid conduit). In some embodiments of the invention, a ventilation conduit is disposed in the system and in operable contact with a fluid conduit along which a droplet travels in the system and/or a humidity reservoir in the system (see, e.g. FIG. 17). Optionally, a surface of a hanging droplet (e.g. one coated with oil) is in direct contact with gases within the humidity reservoir.

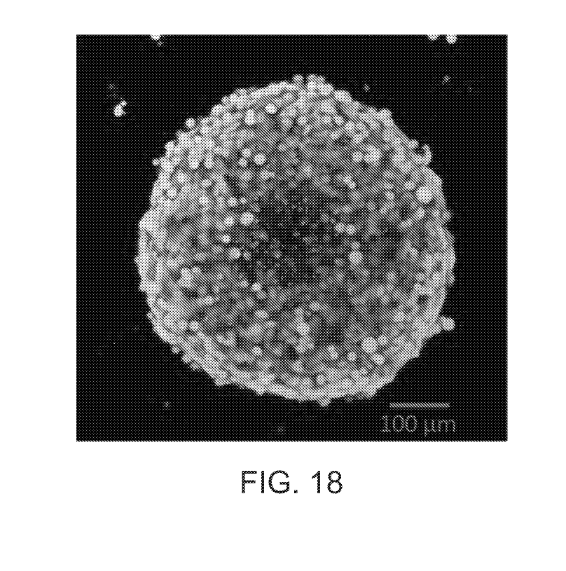

Embodiments of the invention can allow cultured cells within droplet(s) of cell culture media to form spheroid colonies of cells (e.g. mammalian cells) that are at least 2.5×10² μm or at least 5×10² μm in diameter. As shown in FIG. 18, spheroids in one working embodiment of this microfluidic system grew to ˜550 μm in diameter, and further exhibited a center region of necrotic cells, surrounded by a rim of proliferating cells, consistent with spheroids formed by other techniques. Because spheroids with necrotic cores closely resembles the hypoxic regions of tumors, the spheroids generated by the microfluidic systems disclosed herein are observed to mimic physiological conditions observed in tumors in vivo, and in this way overcome problems in studying cell physiology in vitro that can result from in vitro tissue culture conditions being very different from physiological conditions in vivo.

Other illustrative embodiments of the invention comprise methods of forming a spheroid mammalian cell culture within a droplet of cell culture media. These methods typically comprise first providing a microfluidic system as disclosed herein, one which includes for example, a first plate; a second plate parallel to and opposite/facing the first plate; an array of electrodes disposed on the first or second plate; and a well on the first or second plate (e.g. one comprising a hydrophilic surface). Optionally in these methods, the well comprises an open lower end, so that a bottom portion of the droplet of cell culture media is suspended and does not contact a surface; and/or the well comprises a convex surface that contacts and stabilizes droplets of cell culture media. In some embodiments of the invention, the diameter of the well is greater than or equal to 2.4 mm.

In typical methods, artisans place a droplet of cell culture media (e.g. a droplet comprises live mammalian cells and/or agents for modulating the physiology of live mammalian cells) in operable contact with the array of electrodes on the first plate or second plate. Next in these methods, one can then move the droplet of cell culture media along the array of electrodes to the well such that a droplet of cell culture media is drawn into the well by capillary forces. The cultured cells within the droplet of cell culture media can then form a spheroid colony of mammalian cells (e.g. a spheroid at least 5×10² μm in diameters shown in FIG. 18). In some embodiments of the invention, this spheroid is maintained in-situ in the microfluidic system for at least 24 hours or at least 48 hours. In certain embodiments of the invention, this spheroid is maintained in-situ in the microfluidic system for at least 1 or 2 weeks. In certain embodiments, the spheroid is formed in the absence of biologically derived matrices (e.g. collagen or fibrin) and/or the spheroid is formed in the absence of a synthetic hydrogel (e.g. polyacrylamide or PEG). In other embodiments, the spheroid is formed within matrices.

In embodiments of the invention, one can further form a plurality of droplets of liquid cell culture media having a plurality of media conditions; and then move the plurality of droplets through the system using the array of electrodes. Certain embodiments of the invention include the step of coating the droplet of cell culture media with a material that inhibits evaporation (e.g. nonpolar liquid). Optionally in these methods, artisans use a computer processor to facilitate the movement of droplets of liquid along the array of electrodes to the well.

Related embodiments of the invention include methods for delivering an agent to a cell culture using the microfluidic systems disclosed herein (e.g. ones comprising a first plate and a second plate parallel to and opposite the first plate; an array of electrodes disposed on the first or second plate; and a well on the first or second plate, the well comprising a first hanging droplet of cell culture media, wherein the first hanging droplet includes spheroid of mammalian cells). In one instance, these methods comprise of depositing a second hanging droplet of cell culture media on the array of electrodes, wherein the second hanging droplet comprises the agent. The second droplet is then moved along the array of electrodes so that the second hanging droplet is combined with the first hanging droplet, thereby delivering the agent to the cell culture. In other embodiments, these methods comprise of delivering droplets of exogenous agents directly from the reservoirs to a hanging drop. There is no need to form a hanging drop out of the exogenous agents in order to deliver them to a previously existing hanging drop (i.e. a second hanging drop does not need to be formed). Further embodiments and aspects of the invention are discussed below.

In another aspect of the present invention, a microfluidic cell culture system is provided for the creation, maintenance, and/or analysis of three-dimensional, multi-cellular spheroids in an array, as well as the spatially targeted delivery of agents to individual spheroids in the array. In one embodiment, the microfluidic cell culture system is capable of performing all of the various liquid handling steps required for the formation and assaying of scaffold-free three-dimensional cell spheroids on a single platform. The present invention improves upon current digital microfluidic systems and devices by allowing for the culturing of cells in three-dimensions without requiring gels or ECM molecules to encapsulate cells, although such agents may be used if desired. Additionally, the cells may be grown in drops that are not confined within the plates of the microfluidic device. This allows for micro-tissue spheres of at least 0.5×10³ μm in diameter (see, e.g. FIG. 18) to be cultured and analyzed. In certain embodiments, the micro-tissue spheres have a dimension of up to 10³ μm in diameter, which is larger than what is currently possible on existing digital microfluidic devices. In other embodiments, the micro-tissue spheres have a dimension of several millimeters in diameter.

In one embodiment, the microfluidic cell culture system performs any one or all of the various liquid handling protocols necessary for the formation and analysis of three-dimensional, multi-cellular spheroids via a hanging drop technique. With the hanging drop technique, through-holes or wells are incorporated into strategic locations in the bottom plate of the device and droplets of liquid are inserted into these through-holes or wells to form a hanging drop. The ability to freely add, mix, and extract solution from any particular well at any time provides a high degree of control over assay and culture conditions. Thus, the microfluidic cell culture system has the ability to perform the two important functions necessary for hanging drop spheroid cultures: the initiation of hanging drops and the ability to perform medium exchange. Combined, these functions support the formation and maintenance of cell spheroids on the microfluidic device and enable in-situ assaying of individual spheroids.

In one or more embodiments of the present invention, a two-plate microfluidic cell culture system is provided. The microfluidic cell culture system comprises a first plate and a second plate parallel to and opposite the first plate. One or both plates may be transparent, enabling direct visualization and optical spectroscopy. An array of electrodes is patterned on one or both of the parallel plates, which are separated by a defined gap. Typically the gap height is between 50 μm and 500 μm. The electrodes are coated with a dielectric (insulating) material to prevent electrolysis of the liquid to be actuated. Discrete droplets of liquid are dispensed, moved, merged, and mixed through the sequential application of an electric potential to individual electrodes or groups of electrodes. The droplets are driven (actuated) through a combination of electromechanical mechanisms: electrowetting and liquid dielectrophoresis. In various embodiments, one or more elements of the microfluidic cell culture system (e.g. plate, electrode) is transparent to facilitate in situ analysis by microscopy.

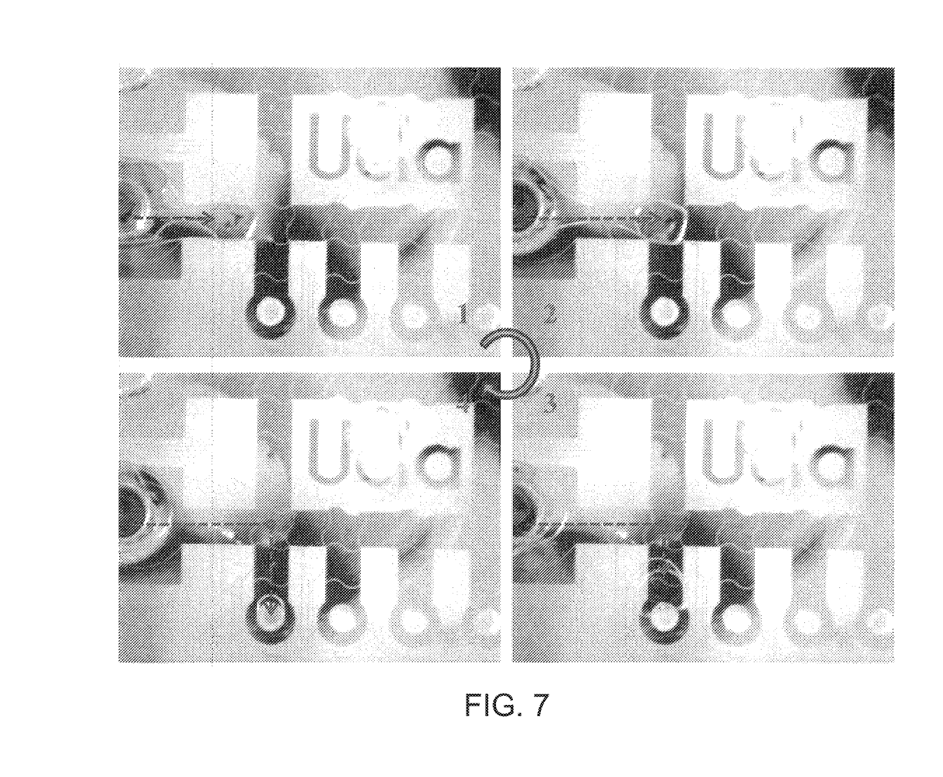

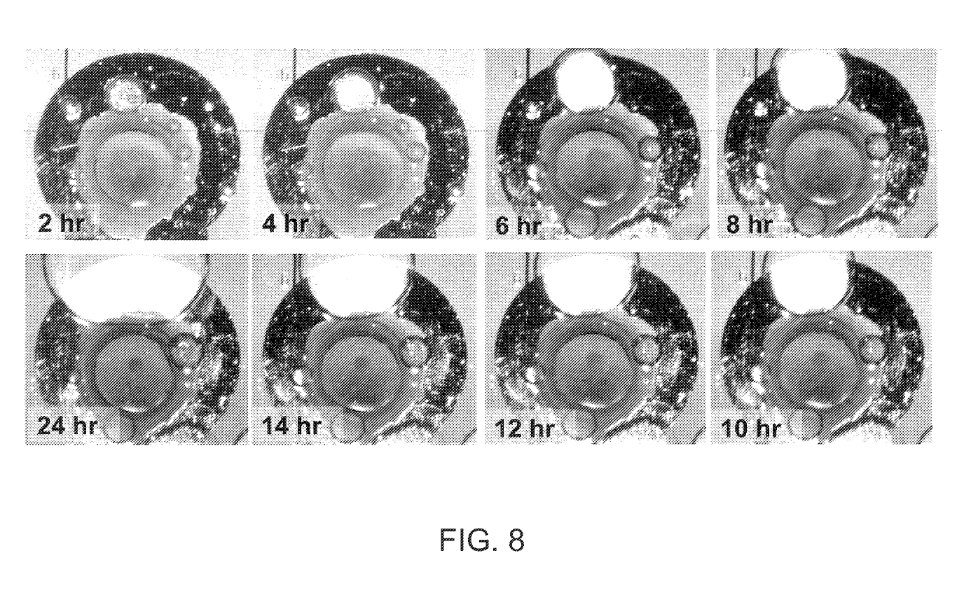

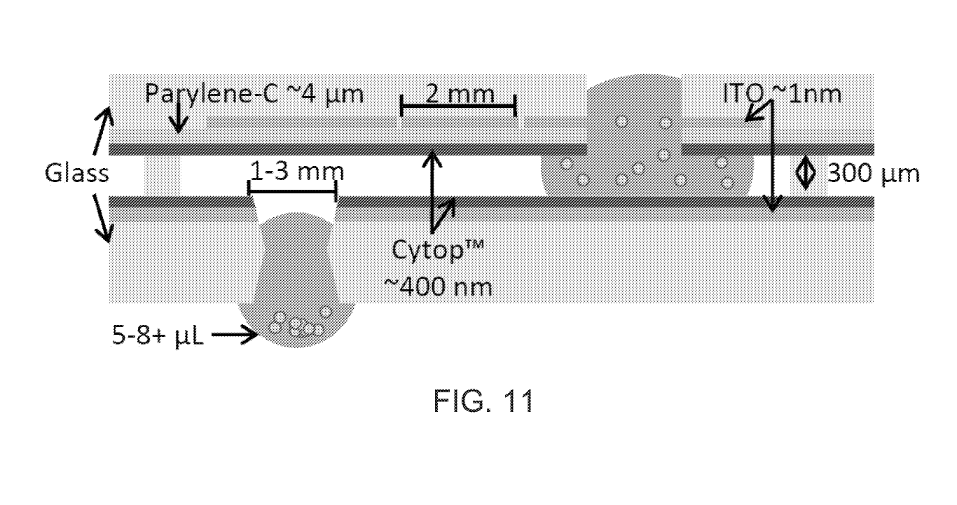

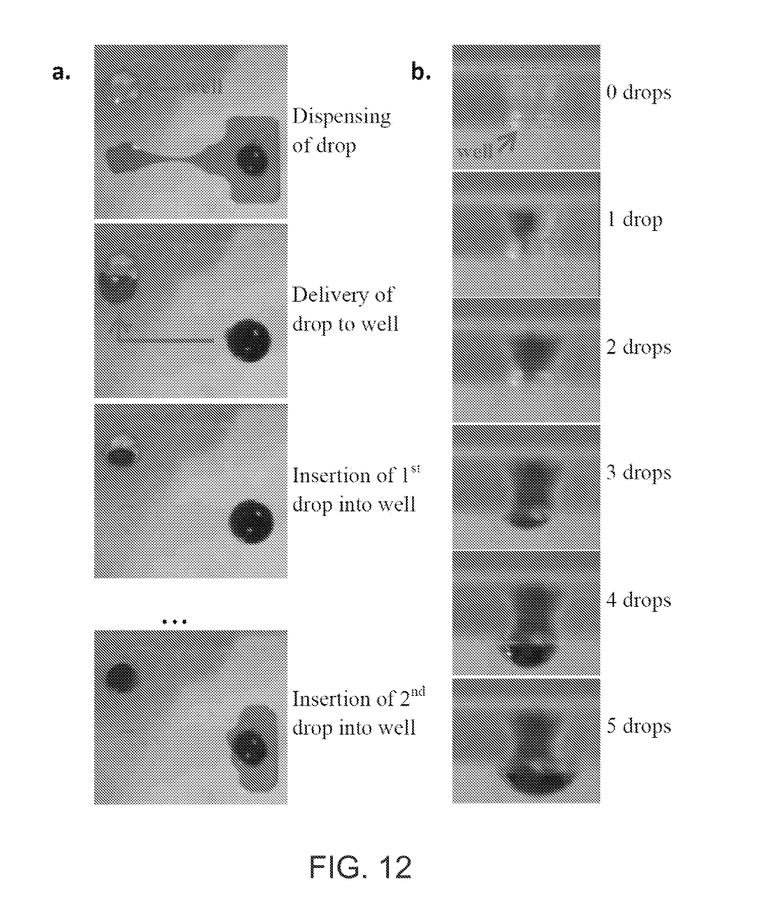

Through-holes or “wells” are fabricated at specific locations on the device such that droplets of liquid can be delivered to each well and drawn into each well by capillary forces (see, e.g. FIGS. 3-6). FIG. 11 shows a schematic of one embodiment of the microfluidic cell culture system along with typical device dimensions. In FIG. 11, drops of liquid are delivered to a through-hole, or ‘well’ in the bottom plate of the device. When the droplet makes contact with the hydrophilic walls of the well, it is pulled into the well spontaneously via capillary forces (FIG. 12a). Adding multiple droplets to a well results in the formation of a concave liquid-air interface that protrudes beneath the bottom of the bottom-plate, similar to a hanging drop (FIG. 12b). Droplets in the wells can be suspended in air or engulfed in a water-immiscible, non-volatile ambient medium such that the bottom of the drop is not touching a solid surface. This allows cells to settle at the concave interface of the drop to form spheroids as part of the hanging drop technique (see, e.g. FIGS. 7, 8). In certain embodiments, the wells are designed such that the Bond number (Bo, a dimensionless parameter describing the ratio of gravitational to surface tension forces) of the system is greater than 0.3, which is within the range where gravitation forces begin to influence the shape of the meniscus (see, e.g. P. Concus, J Fluid Mech, 1968, 34, 481-&; L. Chen, Y. S. Tian et al., Int J Heat Mass Tran, 2006, 49, 4220-4230). Typically, a Bo greater than or equal to 0.3 requires a well diameter greater than or equal to 2.4 mm. In other embodiments, the hanging drops are formed at Bond numbers less than 0.3. Alternatively, round-bottom microwells may be fabricated into the bottom plate such that cells aggregate at the bottom of the wells as part of the microwell technique for spheroid cultures (see, e.g. FIG. 3b).

In another embodiment of the present invention, to simplify device fabrication protocols, the two plates of the digital microfluidic device may be inverted so that the actuating electrodes are in the top-plate of the device and the bottom-plate contains the ground electrode. While both orientations support hanging drop formation, incorporating the wells into the plate containing the actuating electrodes may be more difficult because the wells need to be drilled precisely within the footprint of an electrode, which has the possibility of occasionally resulting in damaged electrodes. Additionally, decoupling the wells and actuating electrodes allows for the actuating top-plate to be removed and replaced in case of a dielectric breakdown, without disrupting the hanging drops in the wells in the bottom-plate. To allow visualization of droplet handling, the actuating electrodes in the top plate may be made from a transparent conductive material, such as indium tin oxide (ITO).

Hanging drops can be formed out of any kind of liquid that can be moved on a digital microfluidic device, including liquids that contain dissolved solutes, or a suspension of solid materials such as cells or beads. Additionally, hanging drops can be made solid by delivering liquids that crosslink into a gel under specific conditions. By forming hanging drops of a cell suspension solution, the digital microfluidic device allows for the formation of multi-cellular spheroids; cells settle at the bottom surface of the hanging drop and form a compact, multi-cellular aggregate over time. Keeping the device at optimal cell culture conditions ensures that the cells can proliferate and maintain viability while in the hanging drops.

Embodiments of the present invention can utilize a variety electrical elements known in the art such as potentiostats (e.g. as shown in FIG. 7 of U.S. Patent Application Publication No. 2012/0283538). Such potentiostats may include an op amp that is connected in an electrical circuit so as to have two inputs: Vset and Vmeasured. Vmeasured is the measured value of the voltage between a reference electrode and a working electrode. Vset, on the other hand, is the optimally desired voltage across the working and reference electrodes. In such embodiments, the voltage between the working and reference electrodes can be controlled by providing a current to the counter electrode.

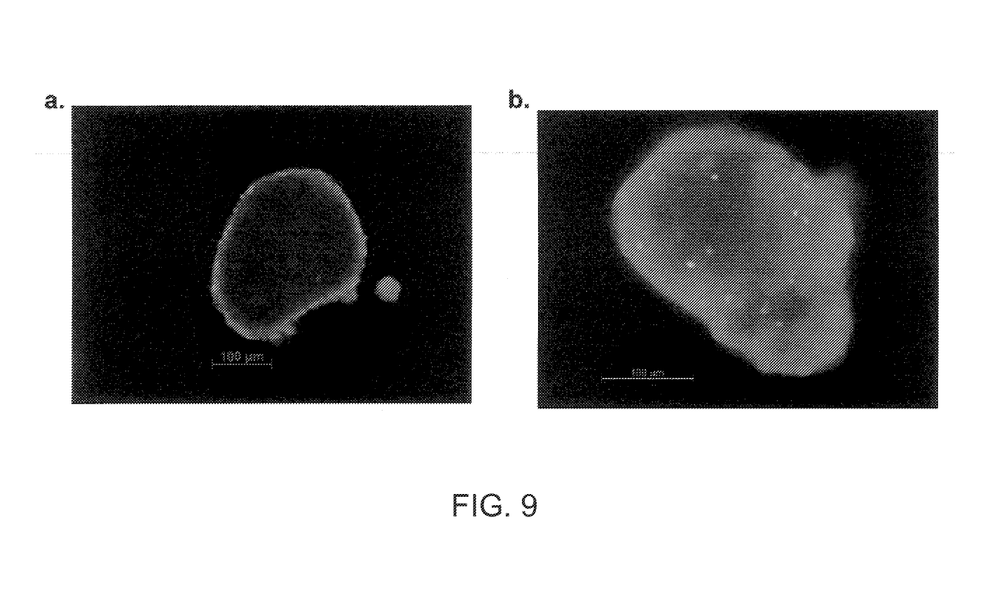

Illustrative experiments have demonstrated the ability of the microfluidic cell culture system to deliver droplets of cell suspension from a reservoir to a well upon which the droplet is spontaneously drawn into the well and anchored within the well, thereby forming a stable hanging drop. These experiments demonstrate the ability to maintain a hanging droplet containing cells at physiological temperature, in-situ, without evaporation for an extended period of time (greater than 24 hours) (see, e.g. FIG. 8). Even in non-optimized systems, cells maintained within a hanging drop on the device aggregate to form three-dimensional clusters over the course of 24 hours and exhibit good viability (see, e.g. FIG. 9). In one embodiment, the microfluidic cell culture system has been found to enable the formation of hanging drops and support the growth of viable spheroids for at least 48 hours of in-situ culture. In other embodiments, by optimizing medium exchange protocols, long-term (greater than 2 week) spheroid culturing and assaying capabilities are possible.

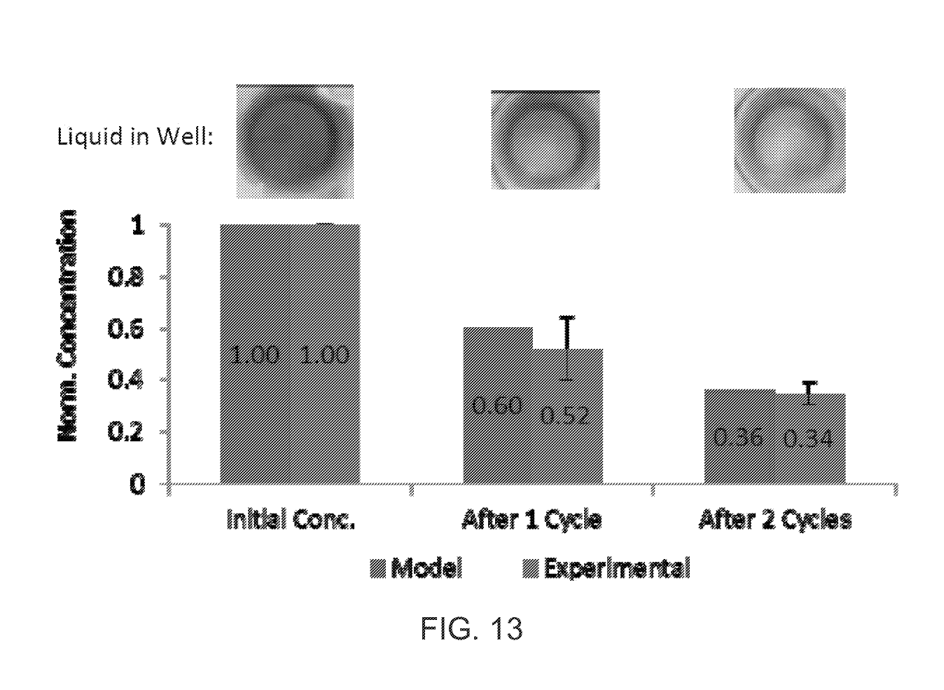

In another aspect of the invention, the microfluidic cell culture system is able to move cell media, protein solutions, cell suspensions, and surfactant solutions. The droplets of solutions required for cell culture and analysis are delivered to and extracted from the wells electromechanically upon application of a voltage. In one certain instance, the voltage is approximately 100V peak-to-peak alternating current (AC). Medium exchange may be performed by extracting drops of spent medium from a hanging drop and replacing it with drops of fresh medium. Repeating the extraction/replacement process sequentially results in a greater degree of medium exchange (see, e.g. FIG. 13). FIG. 13 shows data obtained for one embodiment of the technique for performing medium exchange. Other medium exchange techniques may also be employed in spheroid culture. For example, instead of doing serial dilution of the hanging drop after multiple exchange cycles, a majority of the spent medium within a drop can be extracted initially, and then replaced with fresh medium, without having to go through multiple intermediate steps that require droplet mixing. In certain embodiments, the medium exchange process is performed using electrowetting-driven droplet handling. Typically, cell spheroids require ˜50% medium exchange every 48 hours for optimal growth (see, e.g. J. Friedrich et al., Nat Protoc, 2009, 4, 309-324; in HDP1384 Perfecta3D® 384-Well Hanging Drop Plates Protocol, 3D Biomatrix Inc., 2012). In one exemplary implementation, a method for medium exchange is provided comprising: (1) delivering a fresh drop of medium to a hanging drop, (2) mixing the hanging drop through rapid actuation of the adjacent electrodes, (3) extracting a drop from the hanging drop that is twice the volume of the drop that was initially added, and (4) replacing the extracted drop with a drop of fresh medium. Thus, as an example, assuming an initial hanging drop volume of 8 μl, added drop volume of 2 μl and an extracted drop volume of 4 μl, such a protocol allows for exchange of 40% and 64% of the initial drop volume after one and two cycles, respectively. Using a hanging drop of a standardized dye solution to mimic spent medium and DI water as the ‘fresh’ solution, the degree of exchange may be determined by measuring the change in dye concentration of the hanging drop after multiple exchange cycles using visible spectrophotometry (NanoDrop 2000c, Thermo Scientific). The data (FIG. 13) agree well with the theoretical model, indicating that a medium exchange of greater than 50% can be achieved with one or more exchange cycles.

As an automated, flexible, and low cost platform that allows for completely automated cell spheroid culturing without the need for robotic liquid handling equipment, the microfluidic cell culture system is a powerful and accessible tool for the study of three-dimensional micro-tissues. The microfluidic cell culture system provides an alternative way to grow cell spheroids, which, independently of how they are formed, are better cell models than monolayer cell culture. This not only enhances basic research, but is also extremely valuable in industrial research, particularly within the pharmaceutical industry, where failure rates for drug candidates entering clinical trials are greater than 80% (see, e.g. J. A. DiMasi et al., Clin Pharmacol Ther, 2010, 87, 272-277; J. A. DiMasi et al., Clin Pharmacol Ther, 2013, 94, 329-335; H. Ledford, Nature, 2011, 477, 526-528; K. S. Jayasundara et al., J Rheumatol, 2012, 39, 2066-2070; M. Hay et al., Nat Biotechnol, 2014, 32, 40-51). Such 3D cell models are important in cell-based assays and screens.

The microfluidic cell culture system is capable of supporting the culture of any spheroid-forming cell type or combination of cell types, allowing for the modeling of complex tissues. With this system, spheroids can be cultured under various conditions: e.g., with various media/sera combinations, with bioactive molecules such as ECM proteins, with synthetic biomaterials such as hydrogels, scaffolds, or nanoparticles, in the presence of other biological organisms such as microbes, or exposed to external stimuli such as electric fields or ultraviolet light. The microfluidic cell culture system contains multiple wells to allow for the formation and analysis of multiple spheroids simultaneously. In certain embodiments, the microfluidic cell culture system allows for spheroids ranging in size from 10 to 10³ μm in diameter. In other embodiments, the microfluidic cell culture system allows for spheroids that are several millimeters in diameter.

In addition to enabling the culture of 3D micro-tissues, the microfluidic cell culture system may be used for other various biochemical and biological processes. The microfluidic cell culture system may be used in the automation of any process that utilizes hanging drops. For example, the system may be used for protein crystallization techniques, in-vitro fertilization methods, and in bacterial motility assays (see, e.g. Y. Tang et al., Fertil Steril, 2011, 96, S241-S241; S. W. Potter et al., The Anatomical record, 1985, 211, 48-56; M. A. Dessau et al., J Vis Exp, 2011, DOI: 10.3791/2285; V. Mikol et al., Anal Biochem, 1990, 186, 332-339; P. Kinnunen et al., Small, 2012, 8, 2477-2482; A. Kelman et al., Journal of general microbiology, 1973, 76, 177-188; J. Adler et al., Journal of general microbiology, 1967, 46, 175-184). Because the present invention provides a high level of control over the cellular microenvironment and also allows for in-situ analysis, in one embodiment, the microfluidic cell culture system is used to support the culturing of embryos for in-vitro fertilization (IVF) processes. This embodiment requires minimal handling of cells and a precise culture environment to yield embryos suitable for implantation. In other embodiments, the microfluidic cell culture system is used in academic, industrial, and public sectors for basic research in cellular biology (e.g. to develop novel cell lines, synthetic proteins or genes, drug delivery technologies, cellular imaging methodologies, and biomaterials). The microfluidic cell culture system may also be used by diagnostic laboratories that provide diagnostic services based on the culture and analysis of primary cells and/or bodily fluid samples.

Additionally, the microfluidic cell culture system may be used to support and study: (a) the formation of solid tumors and their sensitivity to biological and chemical agents (e.g., drug candidates); (b) stem cell differentiation, or (c) any biological or physiological system in which a three-dimensional cell model is relevant. An important commercial application is drug screening, since the microfluidic cell culture system is an efficient platform for assessing the effect of a drug on a tissue model. Because multiple spheroids can be created, maintained, and analyzed in an array format, the microfluidic cell culture system can be utilized by pharmaceutical companies to characterize drug uptake and transport in a tissue model (pharmacokinetics), to characterize the effect of drugs on three-dimensional tissue models by monitoring changes in spheroid morphology or secretions, to develop drug delivery technologies, and to characterize cell populations. The microfluidic cell culture system is also useful for validating promising hits from high-throughput drug screens prior to testing the drug candidates in animals and humans. In an exemplary implementation, this microfluidic platform is utilized to study cytokine-induced multi drug resistance mechanisms in a three-dimension human cancer model.

The microfluidic cell culture system provides a number of unique advantages for cell spheroid culturing. In certain embodiments, a computer is used to program the sequence of droplet movements. This allows for automatable and programmable electrowetting-driven liquid handling to form the hanging drops. Exemplary device dimensions and operating parameters are listed in Table 1 below. Automated liquid handling increases throughput and minimizes hands-on time compared to manual spheroid culture techniques. This further reduces variability and human-error in spheroid culture and assay protocols. Additionally, since the microfluidic cell culture system requires no moving parts, minimal consumable use, and low working volumes, the system is a lower-cost, more accessible alternative to existing automated spheroid culture techniques that rely on robotic liquid handling equipment.

Furthermore, the ability to interrogate and address spheroids either individually or in parallel allows for a degree of flexibility in spheroid culturing, treatment, and analysis that is difficult or impossible to achieve with currently available automated methods and systems. This advantage allows information to be gained from individual spheroids that might otherwise be lost due to population averaging—a limitation of massively parallel spheroid culture methods. The microfluidic cell culture system enables in-situ, real-time analysis of individual spheroids, which is not possible using current micromold or massively parallel methods for creating spheroids. Furthermore, less sample and reagent volume is required for culture and analysis, thus reducing costs compared to microplate-based methods.

Additionally, due to the relatively small scale and power requirements of the microfluidic cell culture system compared to robotic liquid handlers, the entire microfluidic system, including the chip and computer control elements, may be packaged into a compact bench-top instrument that may be accommodated in virtually any research environment. Such an instrument provides a less expensive and simpler, more user-friendly approach to automated cell spheroid culturing, making spheroid cultures accessible to almost any research laboratory.

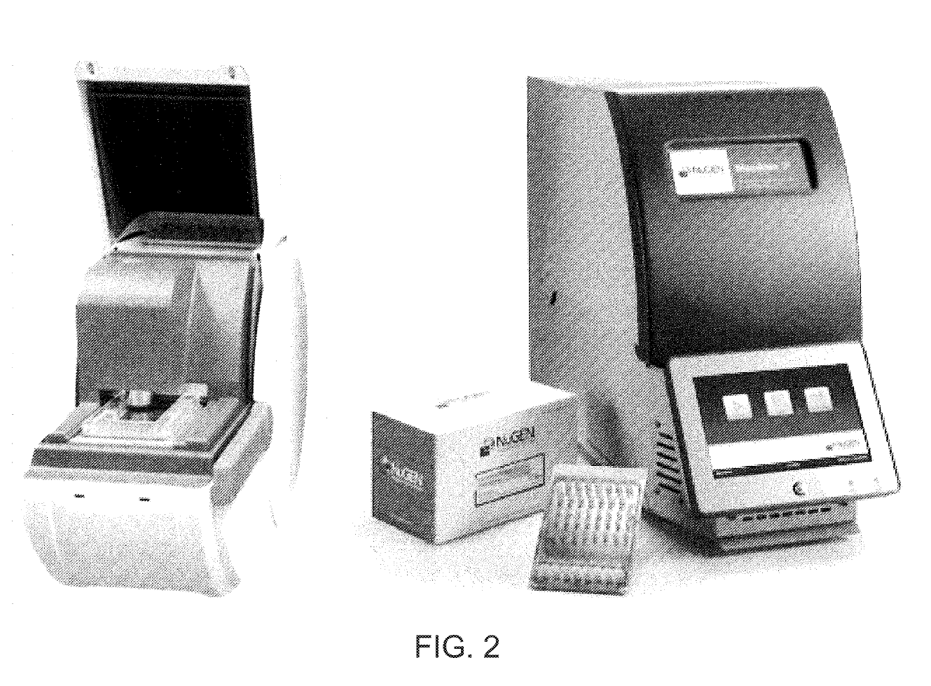

In embodiments of the invention, a microfluidic cell culture system is provided further comprising a benchtop instrument that interfaces with a microfluidic cell culture device and has liquid dispensing components, temperature and humidity control, microscopy capabilities, and/or optical detection components integrated into the instrument. The digital microfluidic device is placed into this instrument and maintained under optimal cell culture conditions in an enclosed environment. The benchtop instrument may be similar to those sold by Advanced Liquid Logic, Inc. and used for digital microfluidic biomolecular sample preparation and analysis (see, e.g. FIG. 2). In specific embodiments, the digital microfluidic device contains multiple locations for cell spheroid culturing and electrode paths leading to/from those locations to allow for the delivery/removal of liquid from the cells. The devices may possess a variety of electrode array patterns and number of spheroid culture sites and are not limited to a single design. The instrument may be connected to a computer or contain a computer processor and an integrated user interface to allow the programming of the droplet manipulation protocols. In one or more embodiments of this invention, the microfluidic device is disposable.

Embodiments of the invention include methods for making the microfluidic cell culture systems disclosed herein. Typically these methods can comprise forming a first plate, forming a second plate parallel to and opposite the first plate, wherein the first or second plate is formed to contain a well, disposing an array of electrodes on the first plate or the second plate. In such methods, the well is disposed on the first or second plate so that when electric potential is applied to the array of electrodes, a droplet of liquid cell culture media within the system moves along the array of electrodes and to the well, so that the droplet of liquid cell culture media is drawn into the well by capillary forces.

As noted above, in typical embodiments of the invention, the liquid manipulations necessary to create, maintain, and analyze cells in hanging drops can be controlled in an automated fashion using conventional computer system elements. FIG. 10 illustrates an exemplary generalized computer system 202 having elements that can be used with embodiments of the present invention. The computer 202 can comprise a general purpose hardware processor 204A and/or a special purpose hardware processor 204B (hereinafter alternatively collectively referred to as processor 204) and a memory 206, such as random access memory (RAM). The computer 202 may be coupled to other devices, including input/output (I/O) devices such as a keyboard 214, a mouse device 216, a potentiostat, a printer 228, etc.

In one embodiment, the computer 202 operates by the general purpose processor 204A performing instructions defined by the computer program 210 under control of an operating system 208 (e.g. instructions to apply a electric potential to an array of electrodes in a manner that allows a droplet of cell culture media to be moved through a microfluidic system). The computer program 210 and/or the operating system 208 may be stored in the memory 206 and may interface with the user and/or other devices to accept input and commands and, based on such input and commands and the instructions defined by the computer program 210 and operating system 208 to provide output and results. Output/results may be presented on the display 222 or provided to another device for presentation or further processing or action. In one embodiment, the display 222 comprises a liquid crystal display (LCD) having a plurality of separately addressable liquid crystals. Each liquid crystal of the display 222 changes to an opaque or translucent state to form a part of the image on the display in response to the data or information generated by the processor 204 from the application of the instructions of the computer program 210 and/or operating system 208 to the input and commands. The image may be provided through a graphical user interface (GUI) module 218A. Although the GUI module 218A is depicted as a separate module, the instructions performing the GUI functions can be resident or distributed in the operating system 208, the computer program 210, or implemented with special purpose memory and processors.

Some or all of the operations performed by the computer 202 according to the computer program 210 instructions may be implemented in a special purpose processor 204B. In this embodiment, some or all of the computer program 210 instructions may be implemented via firmware instructions stored in a read only memory (ROM), a programmable read only memory (PROM) or flash memory in within the special purpose processor 204B or in memory 206. The special purpose processor 204B may also be hardwired through circuit design to perform some or all of the operations to implement the present invention. Further, the special purpose processor 204B may be a hybrid processor, which includes dedicated circuitry for performing a subset of functions, and other circuits for performing more general functions such as responding to computer program instructions. In one embodiment, the special purpose processor is an application specific integrated circuit (ASIC).

In one embodiment, instructions implementing the operating system 208, the computer program 210, and the compiler 212 are tangibly embodied in a computer-readable medium, e.g., data storage device 220, which could include one or more fixed or removable data storage devices, such as a zip drive, floppy disc drive 224, hard drive, CD-ROM drive, tape drive, etc. Further, the operating system 208 and the computer program 210 are comprised of computer program instructions which, when accessed, read and executed by the computer 202, causes the computer 202 to perform the steps necessary to implement and/or use the present invention or to load the program of instructions into a memory, thus creating a special purpose data structure causing the computer to operate as a specially programmed computer executing the method steps described herein. Computer program 210 and/or operating instructions may also be tangibly embodied in memory 206 and/or data communications devices 230, thereby making a computer program product or article of manufacture according to the invention. As such, the terms “article of manufacture,” “program storage device” and “computer program product” as used herein are intended to encompass a computer program accessible from any computer readable device or media.

Table 1 below illustrates typical operating properties for a digital microfluidic device, in accordance with certain embodiments of the present invention.

TABLE 1
Typical operating properties for a digital microfluidic device

| Feature | Typical Dimension Range |
|---|---|
| Device area | 1-20+ sq. in |
| Droplet volume | 0.1-1000 μL |
| Operating voltage | 10-200 V ACpp |
| Operating frequency | DC or 50 kHz AC (Hz to MHz |
| | range possible) |
| Spheroid diameter | 100-1000+ μm |
| Gap height | 50-300+ μm |

### EXAMPLES

### Example 1

### Illustrative Embodiments of the Invention

### Material and Methods

Briefly, to fabricate the microfluidic cell culture system, glass substrates were coated with 1100 Å indium tin oxide (ITO) via sputtering and patterned with electrodes via photolithography and reactive ion etching. For this work, the substrate with the patterned electrodes was used as the top-plate and an un-patterned ITO slide was used as the bottom-plate. Prior to coating with the dielectric, through-holes were manually drilled into specific locations on the bottom-plate using a benchtop drill press and diamond-coated drill bits. Through-holes were also drilled into the footprint of the reservoir electrodes in the top-plate to provide a world-to-chip interface. The top-plates were then coated with 3-4 μm of dielectric polymer parylene-C(Specialty Coating Systems) via vapor deposition. A hydrophobic coating was subsequently applied to both the top and bottom-plates by spin coating ˜300-400 nm of Cytop®. Prior to use, the inside of the wells in the bottom-plate were gently scraped with a diamond-coated drill bit to remove the Cytop® coating on the well walls so as to expose the hydrophilic glass surface.

Analysis of droplet liquid exchange was performed by measuring the absorption of standard dye solutions before and after liquid exchange cycles using a Thermo Scientific NanoDrop 2000c UV-Vis spectrophotometer.

### Preparation of Cell Solutions

Briefly, mouse mesenchymal stem cells (MSCs) at passage 10 were thawed and seeded in polystyrene dishes in growth medium (DMEM, 4 mM L-glutamine, 20% FBS, 100 U/mL P/S solution). Cells were grown to ˜70% confluency and were harvested and re-suspended in spheroid growth medium (Liebovitz L-15, 4 mM L-glutamine, 7.5% FBS, 100 U/mL P/S, 0.04% Pluronic® F-68) at ˜7.5e5 cells/mL for culture on the device.

Prior to use, the devices were sterilized by dipping them in a 70% aqueous ethanol solution and gently drying with compressed air. For device operation, the bottom-plate was placed on an aluminum holding plate with a milled recess to allow hanging drops to form beneath the device. The bottom-plate was sealed on the plate using silicone grease (Dow Corning High Vacuum Grease). The bottom of the recess was enclosed with a glass slide to prevent exposure of the hanging drops to the laboratory environment during drop formation. To minimize evaporation, 1.5 μl droplets of 10 cst silicone oil were pre-loaded into each well prior to the formation of hanging drops. The oil in the wells engulfs the hanging drops upon formation, providing a protective coating against evaporation. Additionally, a small amount of water was placed in the enclosed recess to create a humidified environment. The top-plate was secured to another aluminum plate and was interfaced with the bottom-plate such that particular electrodes in the top-plate aligned with the location of the wells in the bottom-plate. The two plates were separated by a custom designed adhesive silicone spacers (Grace Biolabs, Bend, Oreg.) to create a gap height of 300 μm and were secured using binder clips. Droplets of cell-suspension were added to the reservoir electrodes via through-holes drilled into the top-plate. A schematic of the experimental setup is shown in FIG. 17.

Hanging drop and spheroid formation was achieved by dispensing droplets of cell suspension from the reservoir and moving the droplets to the location of a well. Upon contact with the well, droplets were pulled into the well via capillary forces. Addition of multiple droplets to a well resulted in the formation of hanging drops. Exchange of the medium within the hanging drop was achieved by: (1) delivering a drop of fresh medium to a well, (2) using electrowetting actuation to agitate and mix the drop in the well, (3) extracting a drop from the well of twice the volume of the amount initially delivered, and (4) adding another drop of fresh medium to the well. Devices were kept in an incubator at 37° C. and 95% relative humidity at all times except during liquid handling.

For confocal imaging, spheroids were manually extracted from the device and placed into individual wells in an 8-chambered cover glass (Thermo Scientific™ Nunc™ Lab-Tek™ II Chambered Coverglass) and treated with calcein-am and ethidium homodimer-1 (Life Technologies, LIVE/DEAD® Viability/Cytotoxicity Kit, for mammalian cells). Spheroids were incubated in 2 μM calcein-am and 4 μM ethidium homodimer-1 in Hank's Balanced Salt Solution (HBSS, Life Technologies) for 30 minutes at room temperature, washed with HBSS, and imaged on a Leica TCS SP1 confocal microscope. Spheroid images were constructed by creating a maximum projection of multiple z-plane sections spaced 2-4 μm apart. The percentage of living cells within a spheroid was estimated by counting the number of live (green) and dead (red) cells in 5 different, equally spaced z-planes throughout the spheroid. ImageJ was used for all image analysis.

### Hanging Drop Culture Protocol

A complete hanging drop culture protocol was performed to demonstrate proof-of-principle for a fully automated microfluidic cell spheroid culture system. Droplets of cell suspension (mouse mesenchymal stem cells ATCC: CRL-12424™, 7.5e5 cells/ml) in growth medium (Leibovitz L-15, 7.5% FBS, 100 U/mL penicillin/streptomycin, 4 mM L-glutamine, 0.04% Pluronics® F-68) were delivered to wells to form hanging drops of ˜5-7 μl (˜3750-5250 cells/drop). Leibovitz L-15 medium was used for spheroid culture because it is buffered by phosphates and free base amino acids instead of sodium bicarbonate, which allows cell growth in the absence of a controlled CO₂ atmosphere, which we currently cannot maintain on our digital microfluidic setup. A small amount (˜1-2 μl) of non-volatile, biocompatible oil (sterile filtered, 10 cst silicone oil) was pre-seeded into each well to provide a protective coating against evaporation upon the formation of hanging drops. Devices were kept in an incubator at 37° C. and RH=95% at all times except during liquid handling. During liquid handling, the microfluidic apparatus was kept at ˜37° C. by placing a thin-film polyimide heater in contact with the aluminium device holder. Medium exchange was performed once daily.

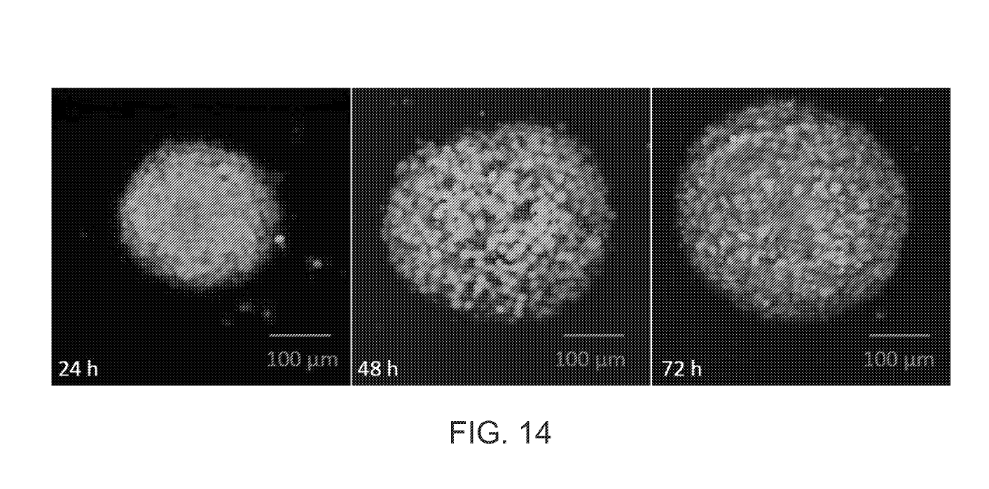

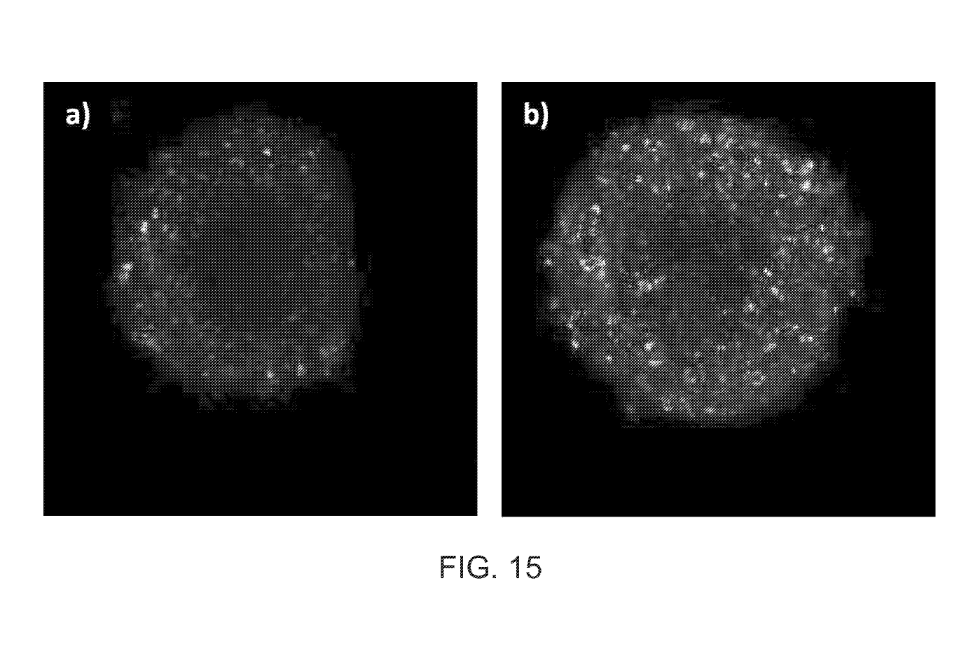

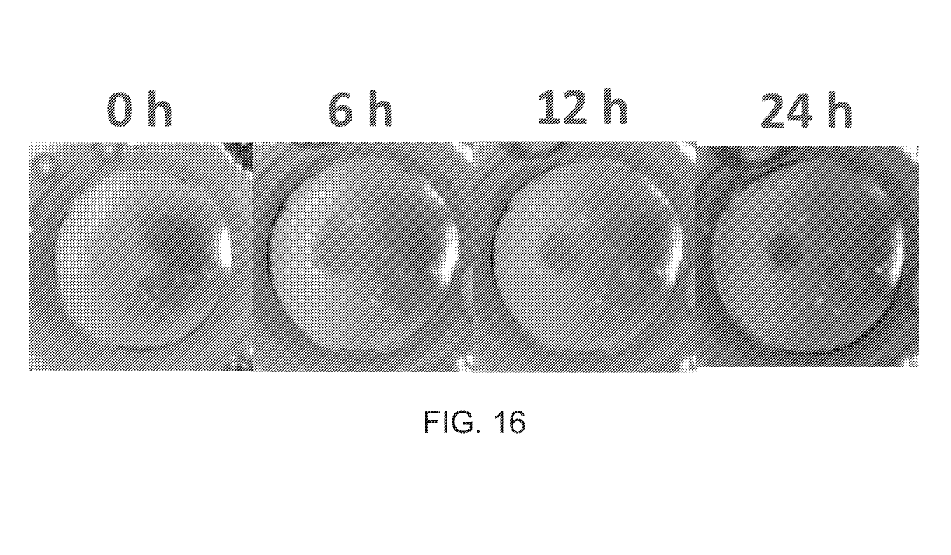

When cell aggregates are kept at optimal culture conditions and medium exchange is performed regularly, the aggregates form compact spheroids that remain viable for up to at least 72 hours in culture. (FIG. 14). Solutions containing any kind of stimulating agent (drug compound, dyes, particles, growth factors, cytokines, exogenous genetic material, etc.) can be delivered to any hanging drop at any time, allowing automated, in-situ assaying of spheroids either individually or in parallel. FIG. 15 shows an example of how the delivery of adipogenic medium to an individual spheroid can induce selective, in-situ differentiation. Because the device is fabricated with transparent electrodes, optical or microscopic analyses can be conducted in-situ. (FIG. 16) Additionally, droplets can be extracted from hanging drops and removed from the device for ex-situ analysis of the cellular supernatant. The device can also be disassembled, allowing spheroids to be extracted for ex-situ handling or analysis.

FIG. 14 shows confocal micrographs of typical spheroids cultured on the microfluidic cell culture system over the course of 72 h using automated sample handling protocols. The spheroids were stained with calcein-AM and ethidium homodimer-1 to indicate living (green) and dead (red) cells, respectively. Counting the number of living and dead cells at various z-planes within the spheroid indicated that the spheroids exhibited >90% cell viability. A seeding density of 7.5e5 cells/mL produced spheroids of up to ˜380 μm after 72 h in culture, which is consistent with spheroid sizes obtained through standard hanging drop techniques (see, e.g. Y. C. Tung et al., Analyst, 2011, 136, 473-478). Spheroids grown on the same device exhibited a size variation of ˜10%.

These results demonstrate that the microfluidic cell culture system can be used to perform fully-automated cell-spheroid culture and has the capability to support the in-situ assaying and analysis of multicellular spheroids. Having established the ability to initiate and maintain viable spheroids in culture and the ability to add, mix, and extract liquid from a well, this microfluidic cell culture system has the ability to provide support for long-term, spheroid-based assays and screens. Because the microfluidic cell culture system provides temporal and spatial control over the handling of discrete drops of liquid, this platform enables extremely flexible assay capabilities as any type of cell, solution, or reagent can be added to or extracted from any particular well at will. This would allow for spheroids to be exposed to drug candidates, differentiation factors, genetic modulators, or other stimuli in a highly controlled fashion. Additionally, genomic or proteomic secretions from spheroids could be extracted for in-situ or ex-situ sample preparation and analysis.

### CONCLUSION

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

All publications, patents, and patent applications cited herein are hereby incorporated by reference in their entirety for all purposes. Nothing here is to be construed as an admission that the inventors are not entitled to antedate the publications by virtue of an earlier priority date or prior date of invention. Further, the actual publication dates may be different from those shown and require independent verification.

1. A microfluidic cell culture system comprising:
a first plate;
a second plate parallel to and opposite the first plate;
an array of electrodes disposed on the first or second plate; and
a well disposed on the first or second plate;
wherein the well is disposed on the first or second plate so that when electric potential is applied to the array of electrodes, a droplet of liquid cell culture media within the system moves along the array of electrodes and to the well, so that the droplet of liquid cell culture media is drawn into the well by capillary forces.

2. The microfluidic system of claim 1, wherein:
the first plate or the second plate comprises a hydrophobic surface disposed on the plate to facilitate movement of the droplet of liquid cell culture media; and
the well comprises a hydrophilic surface.

3. The microfluidic system of claim 2, wherein the well comprises an open lower end, so that a bottom portion of the droplet of liquid cell culture media is suspended and does not contact a surface.

4. The microfluidic system of claim 3, wherein the Bond number of the system is greater than or equal to 0.3.

5. The microfluidic system of claim 1, wherein the well comprises oil that coats the droplet of liquid cell culture media drawn into the well, thereby providing a protective coating against evaporation.

6. The microfluidic system of claim 1, wherein the droplet of liquid cell culture media comprises a spheroid of growing mammalian cells that is from 10 to 10³ μm in diameter.

7. The microfluidic system of claim 1, wherein:
the array of electrodes comprises an actuating electrode and a ground electrode;
and the actuating electrode is disposed on the first plate and the ground electrode is disposed on the second plate.

8. The microfluidic system of claim 1, wherein the array of electrodes is arranged such that a sequential application of an electric potential to the array of electrodes controls the movement of the droplet of liquid cell culture media within the system.

9. The microfluidic system of claim 9, wherein the systems comprises a processor adapted to sequentially apply electric potentials to the array of electrodes.

10. The microfluidic system of claim 1, wherein the system further comprises:
a reservoir adapted to introduce droplets of liquid cell culture media to the system;
a humidity reservoir disposed under the well;
a ventilation conduit through the first plate or the second plate; or
a spacer that separates the first or the second plate at a defined distance.

11. A method of forming a spheroid mammalian cell culture, the method comprising:
(a) providing a microfluidic system comprising:
a first plate;
a second plate parallel to and opposite the first plate;
an array of electrodes disposed on the first or second plate; and
a well on the first or second plate, wherein the well comprises a hydrophilic surface;
(b) placing a droplet of cell culture media in operable contact with the array of electrodes, wherein the cell culture media comprises live mammalian cells;
moving the droplet of cell culture media along the array of electrodes to the well such that the droplet of cell culture media is drawn into the well by capillary forces; and
culturing cells within the droplet of cell culture media so as to form a spheroid of mammalian cells.

12. The method of claim 11, further comprising forming a plurality of droplets of liquid cell culture media having a plurality of media conditions; and
moving the plurality of droplets through the system using the array of electrodes.

13. The method of claim 11, wherein:
the well comprises an open lower end, so that a bottom portion of the droplet of cell culture media is suspended and does not contact a surface; and
the well comprises a convex surface that contacts and stabilizes droplets of cell culture media.

14. The method of claim 11, wherein the spheroid is at least 5×10² μm in diameter.

15. The method of claim 11, wherein the diameter of the well is greater than or equal to 2.4 mm.

16. The method of claim 11, further comprising coating the droplet of cell culture media with a material that inhibits evaporation.

17. The method of claim 11, wherein the spheroid is created in the absence of a synthetic hydrogel composition and/or an exogenously added extracellular matrix composition.

18. The method of claim 11, wherein the spheroid is maintained in-situ in the microfluidic system for at least 24 hours.

19. The method of claim 11, further comprising using a computer processor to move droplets of liquid along the array of electrodes to the well.

20. A method for delivering an agent to a cell culture comprising:
providing a microfluidic system comprising:
a first plate and a second plate parallel to and opposite the first plate;
an array of electrodes disposed on the first or second plate; and
a well on the first or second plate, the well comprising a first hanging droplet of cell culture media, wherein the first hanging droplet includes spheroid of mammalian cells;
depositing a second hanging droplet of cell culture media on the array of electrodes, wherein the second hanging droplet comprises the agent;
moving the second droplet along the array of electrodes so that the second hanging droplet is combined with the first hanging droplet, thereby delivering the agent to the cell culture.
